(19) United States
(12) Patent Application Publication
(10) Pub. No.: US 2013/0296440 A1
(43) Pub. Date: Nov. 7, 2013

(54) PTEROSTILBENE AND CURCUMIN COMBINATION FOR TREATMENT OF OXIDATIVE STRESS AND INFLAMMATION

(71) Applicant: CHROMADEX INC., Irvine, CA (US)

(72) Inventor: Jeremy Bartos, Carlsbad, CA (US)

(73) Assignee: ChromaDex Inc., Irvine, CA (US)

(21) Appl. No.: 13/874,220

(22) Filed: Apr. 30, 2013

Related U.S. Application Data
(60) Provisional application No. 61/640,903, filed on May 1, 2012.

(51) Int. Cl. A61K 31/12; A61K 31/09

(52) U.S. Cl. CPC A61K 31/12; A61K 31/09
USPC 514/679

(57) ABSTRACT

A pharmaceutical composition is provided comprising a therapeutically effective amount of pterostilbene, a therapeutically effective amount of curcumin, and a pharmaceutically acceptable carrier. The embodiments of the pharmaceutical compositions can have lipid lowering properties, or alternatively can have properties that can treat oxidative stress, by decreasing inflammation or inflammatory processes contributing to digestive disorders or cancer. A method of treating an individual for an inflammatory disorder is provided, comprising administering to the individual in need of such treatment a pharmaceutical composition including a therapeutically effective amount of pterostilbene, a therapeutically effective amount of a curcumin, and a pharmaceutically acceptable carrier. The combination containing pterostilbene and curcumin is effective for treatment or prevention of oxidative stress, treatment of digestive disorders including inflammatory bowel disease, and/or to reduce or inhibit inflammation, in both animals and in humans.

This application claims the benefit of earlier filed U.S. Provisional Application No. 61/640,903, filed on May 1, 2012, which is hereby incorporated by reference herein.

### FIELD OF THE INVENTION

A combination of pterostilbene and curcumin is provided. The combination is effective for treatment or prevention of oxidative stress, treatment of digestive disorders including inflammatory bowel disease, and to reduce or inhibit inflammation, in both animals and in humans.

### BACKGROUND

The popular Indian spice turmeric is derived from the rhizomes of Curcuma longa, which is a member of the ginger family (Zingiberaceae). Curcuminoids are polyphenolic compounds that give turmeric its yellow color. Curcumin, a bioactive species having the formula (1), is the principal and prototype curcuminoid.

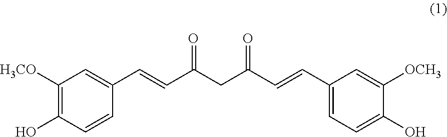 [SMILES: COc1cc(/C=C/C(=O)CC(=O)/C=C/c2ccc(O)c(OC)c2)ccc1O]

Ground turmeric is used as a seasoning and is the main ingredient in curry. Turmeric has been used in the practice of Indian Ayurvedic medicine since 1900 B.C., and in many parts of Southeast Asia it is still used as an alternative medicinal agent for the treatment of a variety of ailments such as stomach ache, flatulence, jaundice, arthritis, sprains, wounds, and skin infections. Curcumin, chemically known as diferuloylmethane, is recognized as the primary biologically active curcuminoid and was first isolated from turmeric almost two centuries ago. Curcumin has recently gained attention as an immune system contributor, with beneficial effects reported in treating arthritis, allergy, asthma, atherosclerosis, heart disease, Alzheimer's disease, cancer, and diabetes.

Management of ulcerative colitis remains problematic despite the wide array of pharmacologic therapies, and compliance issues may reduce the adherence of patients to a drug regimen by 60% at one year. Costs, fear of adverse events, and unknown long-term side effects may distract a patient's attention to a maintenance program. More “natural” programs of therapy, i.e., probiotics, have had less than optimal or even unacceptable results so far.

Pterostilbene (trans-3,5-dimethoxy-4′-hydroxystilbene), originally derived as an extract from blueberries, holds an attraction to patients for its promise of safety combined with efficacy. Other than a welcome reduction in cholesterol levels and blood glucose levels at a higher dosage, this agent's anti-inflammatory (and ultimately anti-neoplastic) properties, particularly when in combination with a synergistic component such as curcumin, may offer a unique benefit to individuals suffering from ulcerative colitis, who are often plagued by the constraints of inflammatory bowel on their lives, and the ever present threat of colorectal cancer.

Pterostilbene is known to activate PPAR-alpha (Rimando, et al., “Pterostilbene, a New Agonist for the Peroxisome Proliferator-Activated Receptor α-Isoform, Lowers Plasma Lipoproteins and Cholesterol in Hypercholesterolemic Hamsters,” J. Agric. Food Chem. (2005) 53:3403-3407), which in the liver leads to increased β-oxidation of fatty acids and decreased triglyceride and very low density lipoprotein (VLDL) synthesis (Fruchart, J.-C.; Duriez, P. Anti-cholesterol agents, new therapeutic approaches. Ann. Pharm. Fr. (2004) 62:3-18). The peroxisome proliferator-activated receptor (PPAR) isoforms belong to the nuclear receptor superfamily of ligand-activated transcription factors which control gene expression by interacting with specific response elements in the promoter region of target genes (Tugwood J D, Aldridge T C, Lambe K G, Macdonald N, Woodyatt N J. Peroxisome proliferator-activated receptors: Structures and function. Ann. N.Y. Acad. Sci. 804, 252, 1996). The primary isoform involved in fatty acid and lipid catabolism and in the activation of genes involved in fatty acid oxidation in the liver is PPAR-alpha (Fruchart J-C, Staels B, Duriez P. PPAR-alpha in lipid and lipoprotein metabolism, vascular inflammation and atherosclerosis. Prog. Exp. Cardiol. 8, 3, 2003). It has been shown that activation of PPAR-alpha in the liver leads to increased oxidation of fatty acids as well as decreased triglyceride and very low density lipoprotein (VLDL) synthesis. This coupled with its ability to induce hepatic apoliporotein A-I and A-II expression, leading to increased plasma HDL cholesterol, makes it a very important target in the cholesterol-lowering field (Gervois P, Torra I P, Fruchart J C, Staels B. Regulation of lipid and lipoprotein metabolism by PPAR activators. Clin. Chem. Lab. Med. 38, 3, 2000). The fibrate family of pharmaceuticals are known PPAR-alpha agonists, and their triglyceride lowering and HDL-cholesterol raising effects are mainly attributed to their activation of PPAR-alpha (Desai R C, Metzger E, Santini C, Meinke P T, Heck J V, Berger J P, MacNaul K L, Cai T, Wright S D, Agrawal A, Moller D E, Sahoo S P. Design and synthesis of potent and subtype-selective PPARalpha agonists. Bioorg. Med. Chem. Lett. 16, 1673, 2006).

Expanding on the story, a report on the activation of PPAR-alpha by resveratrol (Inoue H, Jiang X F, Katayama T, Osada S, Umesono K, Namura S. Brain protection by resveratrol and fenofibrate against stroke requires peroxisome proliferator-activated receptor alpha in mice. Neurosci. Lett. 352, 203, 2003) is the discovery that eventually led to similar studies conducted with pterostilbene (Rimando, 2005). USDA researchers tested the effect of pterostilbene, resveratrol, and ciprofibrate, a member of the fibrate family of pharmaceuticals, on PPAR-alpha activation in H4IIEC3 rat liver cells. Using a luciferase reporter gene construct containing the peroxisome proliferator response element promoter region they found that 100 μM pterostilbene induced PPAR-alpha activation 8-fold over the control and almost 2-fold more than 100 μM of ciprofibrate (while resveratrol was toxic to cells at 100 μM concentrations). At 300 μM pterostilbene concentrations induction grew to 14-fold over the control. This suggested that pterostilbene supplementation should therefore also produce the decreased triglyceride and very low density lipoprotein (VLDL) synthesis and increased plasma HDL cholesterol that results from PPAR-alpha activation by the pharmaceutical fibrate family.

As discussed above, pterostilbene, a natural methylether analog of resveratrol, has been demonstrated to have antioxidant activity similar to that of resveratrol (Rimando et al., (2002) J. Agric. Food Chem. 50:3453-3457; and Stivala et al., (2001) J. Biol. Chem. 276(25):22586-22594). Pterostilbene is present in some small fruits such as grapes (Adrian et al., (2000) J. Agric. Food Chem. 48:6103-6105) and berries of Vaccinium (Vaccinium ashei Reade and Vaccinium stamineum L.) (Rimando et al., (2004) J. Agric. Food Chem. 52:4713-4719) as well as in woody plants (Maurya et al., (1977) J. Nat. Prod. 47:179-181; Amone et al., (1977) J. Chem. Soc. Perkins Trans. 19:2116-2118). However, Rimando, et al. (2004) found that in grapes, pterostilbene is generally found in very minute quantities and may be completely absent. Additionally, a plurality of botanicals contain pterostilbene, including Anogeissus acuminata, Dracaena cochinchinensis, Dracaena loureiri, Guibourtia tessmannii, Pterocarpus macrocarpus, Pterocarpus marsupium, Pterocarpus santalinus, Vaccinium ashei, Vaccinium corymbosum, Vaccinium deliciosum, Vaccinium membranaceum, Vaccinium ovatum, Vaccinium ovalifolium, Vaccinium parviflorum, Vaccinium stamineum, Vaccinium uliginosum, and Vitis vinifrea. Pterostilbene is also found in non-botanical sources such as propolis.

Blueberry species vary in the amount of pterostilbene concentration. It has been reported that a range of 99 ng to 475 ng of pterostilbene can be derived from one gram of lyophilized blueberries.

In view of the above, it would be desirable to provide a pharmaceutical composition having the beneficial properties of pterostilbene and curcumin.

If a way could be found to enhance the lipid lowering properties of pterostilbene (via PPAR-alpha activation pathway) and the antioxidant, chemoprotective and/or anti-inflammatory properties of curcumin in a synergistic manner, this would represent a valuable contribution to the art.

Furthermore, if a way could be found to enhance the known antioxidant, chemoprotective and/or anti-inflammatory properties of pterostilbene and the antioxidant, chemoprotective and/or anti-inflammatory properties of curcumin in a synergistic manner, this would represent a valuable contribution to the art.

Furthermore, if a way could be found to reduce or inhibit inflammatory processes contributing to cancer or digestive diseases by combining pterostilbene with curcumin, this would represent a valuable contribution to the art.

### SUMMARY OF THE INVENTION

A pharmaceutical composition is provided comprising a therapeutically effective amount of pterostilbene, a therapeutically effective amount of curcumin, and a pharmaceutically acceptable carrier. In another embodiment, a pharmaceutical composition comprises a therapeutically effective amount of a combination of pterostilbene and curcumin.

In an embodiment, an anti-inflammatory veterinary composition is provided comprising a therapeutically effective amount of pterostilbene, a therapeutically effective amount of curcumin, and an acceptable carrier.

The embodiments of the pharmaceutical compositions can have lipid lowering properties.

In alternative embodiments, the pharmaceutical compositions can have properties that can treat oxidative stress, by, for example, decreasing inflammation or inflammatory processes contributing to digestive disorders or cancer.

A method of treating an individual for an inflammatory disorder is provided, comprising administering to the individual in need of such treatment a pharmaceutical composition including a therapeutically effective amount of pterostilbene, a therapeutically effective amount of a curcumin, and a pharmaceutically acceptable carrier.

The combination containing pterostilbene and curcumin is effective for treatment or prevention of oxidative stress, treatment of digestive disorders including inflammatory bowel disease, and to reduce or inhibit inflammation, in both animals and in humans.

### BRIEF DESCRIPTION OF THE DRAWINGS

FIG. 1 depicts a proposed metabolic pathway in an embodiment of the present invention, in which curcumin and/or pterostilbene exert synergistic anti-inflammatory effects.

### DETAILED DESCRIPTION

A safe and effective pharmaceutical or nutraceutical composition is described containing pterostilbene and curcumin. In one embodiment, a method of treating an individual for an inflammatory disorder comprises the step of administering to the individual in need of such treatment a pharmaceutical composition comprising a therapeutically effective amount of pterostilbene, a therapeutically effective amount of curcumin, and a pharmaceutically acceptable carrier. An individual subject may be an animal or a human. Animal subjects include large domestic mammals, for example, cattle (or other bovine species), horses, pigs, sheep, goats, other livestock, and the like. Animal subjects may also include smaller domestic mammals, such as, but not limited to, dogs, cats, rabbits, and rodents including rats, mice, hamsters, gerbils, guinea pigs, and the like.

The pterostilbene and curcumin combination is effective for treatment or prevention of oxidative stress, treatment of digestive disorders including inflammatory bowel disease, and to reduce or inhibit inflammation, in either animals or in humans.

In another embodiment, a method of treating an individual for an inflammatory disorder comprises the step of administering to the individual in need of such treatment a pharmaceutical composition comprising a therapeutically effective amount of pterostilbene, a therapeutically effective amount of curcumin, and a pharmaceutically acceptable carrier, wherein inflammation (or a biochemical marker thereof) is decreased. Methods of administering the pharmaceutical compositions as described herein include oral and topical.

In another embodiment, a method of treating an individual for a proliferative disorder or cancer comprises the step of administering to the individual in need of such treatment a pharmaceutical composition comprising a therapeutically effective amount of pterostilbene, a therapeutically effective amount of curcumin, and a pharmaceutically acceptable carrier, wherein the cancer or tumor (or a biochemical cancer or tumor marker thereof) is decreased. Methods of administering the pharmaceutical compositions as described herein include oral and topical.

In another embodiment, a method of reducing oxidative stress in an individual comprises the step of administering to the individual in need of such treatment a pharmaceutical composition comprising a therapeutically effective amount of pterostilbene, a therapeutically effective amount of curcumin, and a pharmaceutically acceptable carrier. Methods of administering the pharmaceutical compositions as described herein include oral and topical.

Ulcerative colitis is a type of inflammatory bowel disease (IBD) that affects the lining of the large intestine (colon) and rectum. It has been observed that daily administration of a standardized turmeric extract significantly diminished symptoms of irritable bowel syndrome. Clinical pilot studies have demonstrated positive effects from curcuminoids in the treatment of Crohn's disease and ulcerative colitis. Animal studies of curcumin show positive effects in ameliorating gastric esophageal reflux disease (GERD). Curcumin has also exerted antiulcerogenic effects in animal models.

Curcuminoids are polyphenolic pigments found in the spice turmeric. The term “turmeric,” as used herein, may be used to refer either to the plant Curcuma longa L. or the spice derived from the rhizomes of the plant. The major curcuminoids are curcumin, having the formula (1),

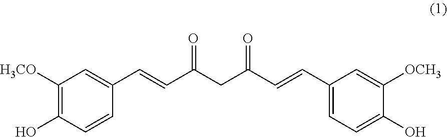 [SMILES: COc1cc(/C=C/C(=O)CC(=O)/C=C/c2ccc(O)c(OC)c2)ccc1O]

demethoxycurcumin, and bisdemethoxycurcumin. These substances comprise 2-6% of Curcuma longa. Curcumin's formal chemical name is (1E,6E)-1,7-Bis(4-hydroxy-3-methoxyphenyl)-1,6-heptadiene-3,5-dione. Curcumin makes up about 70-75% of the total curcuminoids in turmeric, demethoxycurcumin about 10-20%, and bisdemethoxycurcumin generally less than about 5%. Although the present disclosure describes combinations and formulations of curcumin, it is contemplated that suitable analogues or derivatives, and suitable salts, hydrates, solvates, or prodrugs thereof may be useful in the combinations and formulations.

Curcumin may be useful for the treatment of various disorders and diseases, as it possesses strong antioxidant activity. Curcumin is an effective scavenger of reactive oxygen and nitrogen species in vitro (ROS and RNS, respectively). Also, curcumin has demonstrated high anti-inflammatory activity, has been shown to lower histamine levels, and may help alleviate pain and stiffness in the joints from arthritis, bursitis, and tendonitis (Ammon, et al., “Mechanism of anti-inflammatory actions of curcumin and boswellic acids,” J. Ethnopharmacol. (1993) 38:113; and Deodhar, et al. “Preliminary studies on antirheumatic activity of curcumin (diferuloyl methane),” Indian J. Med. Res. (1980) 71:632).

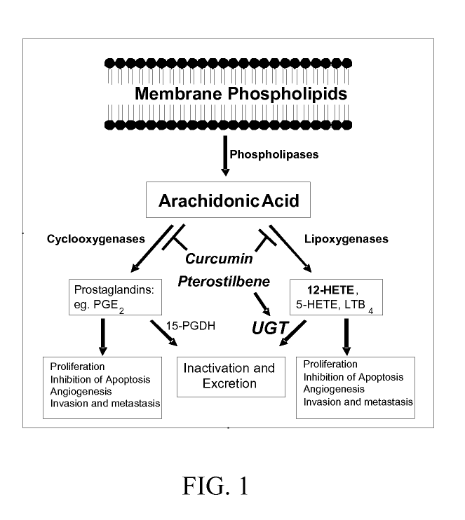

As depicted in FIG. 1, the metabolism of arachidonic acid in cell membranes plays an important role in the inflammatory response by generating potent chemical messengers known as eicosanoids. Membrane phospholipids are hydrolyzed by phospholipase A2 (PLA2), releasing arachidonic acid, which may be metabolized by cyclooxygenases (COX) to form prostaglandins and thromboxanes, or by lipoxygenases (LOX) to form leukotrienes. Curcumin has been found to inhibit PLA2, COX-2, and 5-LOX activities in cultured cells (Hong, et al. “Modulation of arachidonic acid metabolism by curcumin and related beta-diketone derivatives: effects on cytosolic phospholipase A(2), cyclooxygenases and 5-lipoxygenase,” Carcinogenesis (2004) 25(9):1671-1679; and C. V. Rao, “Regulation of COX and LOX by curcumin,” Adv. Exp. Med. Biol. (2007) 595:213-226). Although curcumin inhibited the catalytic activity of 5-LOX directly, it inhibited PLA2 by preventing its phosphorylation and COX-2 mainly by inhibiting its transcription. As a COX-2 inhibitor, curcumin down-regulates the expression of this enzyme, which is linked with most types of inflammations (Zhang, et al., “Curcumin inhibits cyclooxygenase-2 transcription in bile acid and phorbol ester-treated human gastrointestinal epithelial cells,” Carcinogenesis (1999) 20:445; and Tunstall, et al., “Cyclooxygenase-2 expression and oxidative DNA adducts in murine intestinal adenomas: Modification by dietary curcumin and implications for clinical trials,” Eur. J. Cancer (2006) 42:415). Nuclear factor-kappa B (NF-kB) is a transcription factor that binds DNA and enhances the transcription of the COX-2 gene as well as other pro-inflammatory genes, such as inducible nitric oxide synthase (iNOS). In inflammatory cells, such as macrophages, iNOS catalyzes the synthesis of nitric oxide, which can react with superoxide to form peroxynitrite, a reactive nitrogen species that can damage proteins and DNA. Curcumin has been found to inhibit NF-kB-dependent gene transcription, and thus inhibit the induction of COX-2 and iNOS expression in cell culture and in animal studies.

Furthermore, without being bound by theory, the possible anticarcinogenic activity of curcumin may be accounted for by several of the mechanisms as discussed above. Again referring to FIG. 1, curcumin may suppress angiogenesis and induce apoptosis and tumor suppressor genes (Pan, et al., “Induction of apoptosis by garcinol and curcumin through cytochrome c release and activation of caspases in human leukemia HL-60 cells, “J. Agric. Food Chem. (2001) 49:1464; and Liontas, et al., “Curcumin and resveratrol induce apoptosis and nuclear translocation and activation of p53 in human neuroblastoma,” Anticancer Res. (2004) 24:987). The ability of curcumin to induce apoptosis in cultured cancer cells by several different mechanisms has generated scientific interest in potential uses for curcumin to prevent some types of cancer. Oral curcumin administration has been found to inhibit the development of chemically-induced cancer in animal models of oral, stomach, liver, and, in particular, colon cancer (Rao, et al., “Chemoprevention of colon carcinogenesis by dietary curcumin, a naturally occurring plant phenolic compound,” Cancer Res. (1995) 55(2):259-266; and Kawamori, et al., “Chemopreventive effect of curcumin, a naturally occurring anti-inflammatory agent, during the promotion/progression stages of colon cancer,” Cancer Res. (1999) 59(3):597-601). These findings, among others, suggest that oral curcumin is more likely to be effective as a therapeutic agent in cancers of the gastrointestinal (GI) tract then other tissues.

Cancerous cells invade normal tissue with the aid of enzymes called matrix metalloproteinases. Curcumin has been found to inhibit the activity of several matrix metalloproteinases in cell culture studies. To fuel their rapid growth, invasive tumors must also develop new blood vessels by a process known as angiogenesis. As depicted in FIG. 1, curcumin has been found to inhibit angiogenesis in cultured vascular endothelial cells and in an animal model (Arbiser, et al., “Curcumin is an in vivo inhibitor of angiogenesis,” Mol. Med. (1998) 4(6):376-383.).

Several studies have been performed to demonstrate the safety of curcumin. Curcumin doses of 1.8 g/kg/day administered to rats and 0.9 g/kg/day administered to monkeys showed no toxicity or adverse events. A phase I clinical trial on humans with high-risk or premalignant lesions was carried out to determine associated toxicity with high dose curcumin administration. Subjects were administered up to 8000 mg of curcumin per day with no toxic effects experienced (Cheng, et al., “Phase I clinical trial of curcumin, a chemopreventive agent, in patients with high-risk or pre-malignant lesions,” Anticancer Res. (2001) 21:2895). Since then, several clinical studies have used doses of pure curcumin in the 500-2000 mg/day range with no reports of toxic effects or adverse outcomes in humans (Hanai, et al., “Curcumin maintenance therapy for ulcerative colitis: randomized, multicenter, double-blind, placebo-controlled trial,” Clin. Gastroenterol. Hepatol. (2006) 4:1502). Finished products containing curcumin can include 500-1000 mg of curcumin per serving.

In embodiments of the present invention, curcumin may be used in a range from about 100 mg to about 2000 mg per serving or dose. In other embodiments, curcumin may be used in a range up to about 5 g, in particular for the treatment of large animal species, e.g., horses or cattle.

With respect to the uses of curcumin as a therapeutic or nutraceutical agent in the treatment of conditions selected from inflammation, inflammatory or digestive disorders, and cancer, it is expected that combination with pterostilbene will provide synergistic effects. Other synergistic effects using combinations of curcumin and pterostilbene may include: improvement of blood circulation and prevention of atherosclerosis or other vascular diseases; wound healing; cholesterol lowering (as discussed above for pterostilbene); lowering of blood glucose levels and treatments for diabetes; antidepressant activity; and antimicrobial activity, included, but not limited to, antiviral, antibacterial, and antifungal uses.

Curcumin is minimally water soluble, and therefore suffers from poor bioavailability. It is therefore contemplated that encapsulated forms of curcumin may be used in embodiments of the present invention. Certain suitable encapsulated forms include liposomes, micellar emulsions, microemulsions, and the like. Suitable surfactants, emulsifiers, or wetting agents may be used as appropriate in order to construct or formulate the bioactive components as described herein. Conventional liposomal and/or emulsion systems may be used. Methods of administration include oral and intravenous delivery. It is to be understood that these delivery systems may be applied to curcumin, or alternatively, to the combination of curcumin with pterostilbene.

In contrast, pterostilbene (3,5-dimethoxy-4′-hydroxy-trans-stilbene) is an orally bioavailable compound with a half life t_{1/2} of about 74-105 minutes in blood. By comparison, resveratrol has poor bioavailability, and is readily metabolized by UGTs leading to a much shorter half life (t_{1/2} about 10-14 minutes in blood), which hinders its effectiveness as a chemopreventive agent.

The effects of pterostilbene on PPAR-alpha activation are well-established. The peroxisome proliferator-activated receptor (PPAR) isoforms belong to the nuclear receptor superfamily of ligand-activated transcription factors which control gene expression by interacting with specific response elements in the promoter region of target genes. The primary isoform involved in fatty acid and lipid catabolism and in the activation of genes involved in fatty acid oxidation in the liver is PPAR-alpha. It has been shown that activation of PPAR-alpha in the liver leads to increased oxidation of fatty acids as well as decreased triglyceride and very low density lipoprotein (VLDL) synthesis (Fruchart, et al., Ann. Pharm. Fr. (2004) 62:3). This activation, coupled with its ability to induce hepatic apoliporotein A-I and A-II expression, leading to increased plasma HDL cholesterol, makes it a very important target in the cholesterol-lowering field (Gervois, et al., Clin. Chem. Lab. Med. (2000) 38:3). Comparatively, the fibrate family of pharmaceuticals are known PPAR-alpha agonists, and their triglyceride lowering and HDL-cholesterol raising effects are mainly attributed to their activation of PPAR-alpha (Desai, et al., Bioorg. Med. Chem. Lett. (2006) 16:1673).

Dyslipidemias are disorders of lipoprotein metabolism, including lipoprotein overproduction or deficiency. These disorders may be manifested by elevation of the serum total cholesterol (TC), low-density lipoprotein (LDL) cholesterol and triglyceride concentrations, and/or a decrease in the high-density lipoprotein (HDL) cholesterol concentration. Very low-density lipoprotein (VLDL) and total lipoprotein may also be affected.

In certain embodiments of the present invention, the decreased lipid levels may be expressed as a reduction in blood plasma or serum selected from total cholesterol (TC), low-density lipoprotein cholesterol (LDL-C), triglycerides (TG), and the ratio of low-density lipoprotein cholesterol to high-density lipoprotein cholesterol (LDL-C/HDL-C). Measurement and diagnostic determination of blood levels of the aforementioned lipid components in various animal species, mammals, and human individuals is well known in the art.

The UDP-glucuronosyltransferases (UGTs) catalyze the transfer of glucuronic acid from a high-energy cofactor, UDP-glucuronic acid, to a xenobiotic, drug, or endogenous substrate containing an available nucleophilic moiety such as a hydroxyl, carboxyl, amino, or thiol group. The UGTs are Phase II biotransformation enzymes predominantly expressed in liver and intestine, and are membrane-bound enzymes localized on the luminal surface of the endoplasmic reticulum. Relative to the parent substrate, the end-products of glucuronidation are typically more polar and better suited for excretion and elimination through the urine or bile.

Endogenous UGT substrates include bilirubin, neutral steroids, bile acids, fatty acids, and retinoids. Xenobiotic UGT substrates range from environmental toxicants such as benzo[a]pyrene to common pharmaceuticals such as acetaminophen and other NSAIDs. Individual UGT isoforms display distinctive patterns of substrate specificity and inducible regulation. Different UGTs are expressed in a species- and tissue-specific manner. The two major UGT gene families are UGT1 and UGT2.

PPARs are known to be involved in the regulation of UGTs. In particular, the UGTs are targets of PPAR-alpha, as opposed to PPAR-gamma. Resveratrol, a known PPAR-gamma activator, has shown some induction of UGTs, but is not a strong activator of PPAR-alpha. In one study of a group of similar stilbenes, only pterostilbene activated PPAR-alpha, in a manner similar to ciprofibrate (Rimando, et al., J. Agric. Food Chem. (2005) 53:3403-3407). Pterostilbene is not thought to be a strong activator of PPAR-gamma. This sub-type selectivity is thought to be advantageous in the present approach to activate or induce expression of UGTs using pterostilbene, as depicted in FIG. 1. Thus, the enhanced lipid lowering properties of pterostilbene are mediated by these inter-related pathways involving PPAR-alpha activation.

In addition, pterostilbene can be useful in the treatment or prevention of vascular disease. Pterostilbene has been shown to inhibit the proliferation of rat smooth muscle cells in vitro which gives it potential as an anti-proliferative agent for the treatment of atherosclerosis (Park, et al., Vascul. Pharmacol. (2010) 53: 61).

The presence of chronic inflammatory conditions in the colonic environment has been implicated in the development of colorectal cancer, and treatment regimens against inflammatory markers have reduced the risk of colon cancer. Pterostilbene has been shown to reduce the levels of pro-inflammatory cytokine markers such as COX-2, TNF-alpha, IL-4, IL-1-beta, and iNOS both in vitro and in vivo. (See, for example, Hougee, et al., Planta Med. (2005) 71(5):387-392; Paul, et al., Cancer Prey. Res. (Phila). (2009) 2(7):650-657; Paul, et al., Carcinogenesis (2010) 31(7): 1272-1278, each incorporated herein by reference.)

Based on the above discussion, and without intending to be bound by theory, it can be readily appreciated that pterostilbene and curcumin share several advantageous biochemical pathways which may be complementary, if not synergistic, for the purpose of preventing or treating various disorders. See, FIG. 1. It is expected that combinations or formulations including both curcumin and pterostilbene will possess unexpected advantageous properties that exceed or improve the results using either agent in isolation. It is further expected that combinations or formulations including both curcumin and pterostilbene will possess unexpected advantageous properties that exceed or improve the results using either agent administered separately, or at different times.

With respect to Phase I metabolism, pterostilbene is also well known as an inhibitor of cytochrome P450 (R. Mikstacka, et al., Mol. Nutr. Food Res. (2007) 51:517). Pterostilbene has been shown to inhibit CYP1A1 and CYP1B1 isozymes (R. Mikstacka, et al., “Inhibition of human recombinant cytochromes P450 CYP1A1 and CYP1B1 by trans-resveratrol methyl ethers,” Mol. Nutr. Food Res. (2007) 51:517), but not CYP2E1 (R. Mikstacka, et al., “Effect of natural analogues of trans-resveratrol on cytochromes P4501A2 and 2E1 catalytic activities,” Xenobiotica (2006) 36:269).

In another embodiment, a combination of a therapeutically effective amount of pterostilbene and a therapeutically effective amount of curcumin can be used for treatment of dyslipidemias. In another embodiment, a combination of a therapeutically effective amount of pterostilbene and a therapeutically effective amount of curcumin can be used for lipid lowering, for example, reduction of endogenous cholesterol synthesis. It is expected that the combination of pterostilbene and curcumin will provide synergistic effects with regard to treating dyslipidemias and lipid lowering.

In another embodiment, a combination of a therapeutically effective amount of pterostilbene and a therapeutically effective amount of curcumin can be used for reducing the levels of a pro-inflammatory cytokine It is expected that the combination of pterostilbene and curcumin will provide synergistic effects with regard to treating, preventing, and/or reducing inflammation.

Curcumin, 99% Dietary Supplement (DS) grade, is available from ChromaDex, Inc. (Irvine, Calif.).

In one embodiment, curcumin can be provided in daily dosages of from about 50 mg to about 2000 mg, in particular in a human patient, for example. Another suitable dosage range is from about 100 mg to about 2000 mg daily. Another suitable dosage range is from about 50 mg to about 1000 mg daily. Another suitable dosage range is from about 250 mg to about 1000 mg daily. Another suitable dosage range is from about 500 mg to about 1000 mg daily. The daily dose of curcumin may be administered in one or more servings.

The following daily dose ranges of curcumin may be useful in certain animals, i.e., for a veterinary use. Cats: 10-150 mg; dogs: 10-300 mg; horses: 1000-5000 mg.

Pterostilbene (99% purity) is commercially available from ChromaDex, Inc. (Irvine, Calif.).

In one embodiment, pterostilbene can be provided in daily dosages of from about 5 mg to about 500 mg, in particular in a human patient, for example. Another suitable dosage range is from about 10 mg to about 500 mg daily. Another suitable dosage range is from about 25 mg to about 500 mg daily. Another suitable dosage range is from about 50 mg to about 250 mg daily. Another suitable dosage range is from about 50 mg to about 150 mg daily. Another suitable dosage range is from about 50 mg to about 100 mg daily. A particularly suitable dosage is about 100 mg administered daily. The daily dose of pterostilbene may be administered in one or more servings.

The following daily dose ranges of pterostilbene may be useful in certain animals. Cats: 1-50 mg, dogs: 1-150 mg, horses: 100-1500 mg.

The dosages of the curcumin are expected to be particularly effective when used in combination with pterostilbene in solid or liquid form, or as a blend. It is expected that the combination of pterostilbene and curcumin will provide synergistic effects with regard to treatment of inflammation and reduction of biological markers of inflammation and/or oxidative stress. The pterostilbene/curcumin combinations are useful for preventing, alleviating, or treating inflammatory or digestive disorders such as, but not limited to, inflammatory bowel disease, gastric esophageal reflux disease (GERD), and ulcerative colitis. Other inflammatory conditions susceptible to such treatment include: Crohn's disease, laminitis, arthritis, sarcoidosis, acne, periodontitis, atherosclerosis, and the like. The pterostilbene/curcumin combinations are useful for preventing, alleviating, or treating proliferative disorders such as, but not limited to, stomach cancer, pancreatic cancer, oral cancer, liver cancer, pre-cancerous polyps, colorectal adenomas (polyps), colon cancer, colorectal cancer, and other cancers of the gastrointestinal tract.

Also contemplated are pterostilbene/curcumin combinations useful for treatment of disorders related to or caused by oxidative stress, such as suppression of oxidative DNA damage and modulation of apoptosis. Oxidative stress has been implicated as a potential contributing factor in neurodegenerative diseases and aging. Thus, it has been hypothesized that oxidative stress may play a role, or exacerbate symptoms, in conditions such as Alzheimer's disease, Parkinson's disease, and other neurodegenerative diseases. Oxidative stress may be involved in age-related development of cancer. The reactive species produced in oxidative stress can cause direct damage to the DNA and are therefore mutagenic, and may also suppress apoptosis and promote proliferation, invasiveness and metastasis. Oxidative stress is thought to be linked to certain cardiovascular diseases, since oxidation of LDL in the vascular endothelium is a precursor to plaque formation. Oxidative stress also plays a role in the ischemic cascade due to oxygen reperfusion injury following hypoxia. This cascade includes both strokes and heart attacks.

In another embodiment, a pharmaceutical composition comprises a therapeutically effective amount of a combination of pterostilbene and curcumin. Due to expected synergy of the components, the pharmaceutical composition of the combination of pterostilbene and curcumin may provide a therapeutic effect even where the individual components may not provide the therapeutic effect.

In one embodiment, a suitable ratio of pterostilbene to curcumin is about 1:1 weight/weight. Other useful ratios of pterostilbene to curcumin (both in solid form before mixing or other formulation) include 1:2, 1:3, 2:3, 1:4, 1:5, or 1:10 weight/weight. The preferred pterostilbene/curcumin ratios as described are generally substantially maintained in the mixed, formulated, or finished products.

A composition in accordance with the present invention containing pterostilbene, or derivatives thereof, or a pharmaceutically acceptable salt of pterostilbene, can be prepared by conventional procedures for blending and mixing compounds. In another embodiment, pterostilbene in combination with curcumin, a derivative, or salt thereof, can be prepared by conventional procedures for blending and mixing compounds. The composition may optionally include an excipient, most preferably a pharmaceutical excipient. Compositions containing an excipient and incorporating the pterostilbene can be prepared by procedures known in the art. Optionally, the composition can include one or more adjuvants, excipients, carriers, buffers, diluents, and/or other customary pharmaceutical auxiliaries. For example, pterostilbene can be formulated into tablets, capsules, softgels, powders, suspensions, solutions for oral administration and solutions for parenteral administration including intravenous, intradermal, intramuscular, and subcutaneous administration, and into solutions for application onto patches for transdermal application with common and conventional carriers, binders, diluents, and excipients.

The nutraceutical compositions of the present invention may be administered in combination with a nutraceutically acceptable carrier. The active ingredients in such formulations may comprise from 1% by weight to 99% by weight, or alternatively, 0.1% by weight to 99.9% by weight. “Nutraceutically acceptable carrier” means any carrier, diluent or excipient that is compatible with the other ingredients of the formulation and not deleterious to the user. Useful excipients include microcrystalline cellulose, magnesium stearate, calcium stearate, any acceptable sugar (e.g., mannitol, xylitol), and for cosmetic use an oil-base is preferred.

The pharmaceutical compositions of the present invention may be administered in combination with a pharmaceutically acceptable carrier. The active ingredients in such formulations may comprise from 1% by weight to 99% by weight, or alternatively, 0.1% by weight to 99.9% by weight. “Pharmaceutically acceptable carrier” means any carrier, diluent or excipient that is compatible with the other ingredients of the formulation and not deleterious to the user.

Delivery System

Suitable dosage forms include tablets, capsules, softgels, oil-based matrices, solutions, suspensions, powders, gums, and confectionaries. Sublingual delivery systems include, but are not limited to, dissolvable tabs under and on the tongue, liquid drops, and beverages. Edible films, hydrophilic polymers, oral dissolvable films or oral dissolvable strips can be used. Other useful delivery systems comprise oral or nasal sprays or inhalers, and the like.

For oral administration, pterostilbene and/or pterostilbene in combination with a curcumin may be further combined with one or more solid inactive ingredients for the preparation of tablets, capsules, pills, softgels, powders, granules or other suitable dosage forms. For example, the active agent may be combined with at least one excipient such as fillers, binders, humectants, disintegrating agents, solution retarders, absorption accelerators, wetting agents, absorbents, or lubricating agents. Other useful excipients include magnesium stearate, calcium stearate, mannitol, xylitol, sweeteners, starch, carboxymethylcellulose, microcrystalline cellulose, silica, gelatin, silicon dioxide, and the like.

The components of the invention, together with a conventional adjuvant, carrier, or diluent, may thus be placed into the form of pharmaceutical compositions and unit dosages thereof. Such forms include solids, and in particular tablets, filled capsules, powder and pellet forms, and liquids, in particular aqueous or non-aqueous solutions, suspensions, emulsions, elixirs, and capsules filled with the same, all for oral use, suppositories for rectal administration, and sterile injectable solutions for parenteral use. Such pharmaceutical compositions and unit dosage forms thereof many comprise conventional ingredients in conventional proportions, with or without additional active compounds or principles, and such unit dosage forms may contain any suitable effective amount of the active ingredient commensurate with the intended daily dosage range to be employed.

The components of the present invention can be administered in a wide variety of oral and parenteral dosage forms. It will be obvious to those skilled in the art that the following dosage forms may comprise, as the active component, either a chemical compound of the invention or a pharmaceutically acceptable salt, solvate, hydrate, or prodrug of a chemical compound of the invention.

For preparing pharmaceutical compositions from a chemical compound of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid, which is in a mixture with the finely divided active component. In tablets, the active component is mixed with the carrier having the necessary binding capacity in suitable proportions and compacted in the shape and size desired.

For example, the powders and tablets may contain from five or ten to about seventy percent of the active compound(s). Suitable carriers are magnesium carbonate, magnesium state, talc, sugar, lactose, pectin, dextrin, starch, gelatin, tragacanth, methylcellulose, sodium carboxymethlycellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, are included. Tablets, powders, capsules, pills, cachets, and lozenges can be used as solid forms suitable for oral administration.

Liquid preparations include solutions, suspensions, and emulsions, for example, water or water-propylene glycol solutions. For example, parenteral injection liquid preparations can be formulated as solutions in aqueous polyethylene glycol solution. The chemical compounds, mixtures, or blends according to the present invention may thus be formulated for parenteral administration (e.g. by injection, for example bolus injection or continuous infusion) and may be presented in unit dose for in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilising and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

Aqueous solutions suitable for oral use can be prepared by dissolving the active component in water and adding suitable colorants, flavors, stabilizing and thickening agents, as desired. Aqueous suspensions suitable for oral use can be made by dispersing the finely divided active component in water with viscous material, such as natural or synthetic gums, resins, methylcellulose, sodium carboxymethylcellulose, or other well known suspending agents.

Compositions suitable for topical administration in the mouth includes lozenges comprising the active agent in a flavored base, usually sucrose and acacia or tragacanth; pastilles comprising the active ingredient in an inert base such as gelatin and glycerine or sucrose and acacia; and mouthwashes comprising the active ingredient in suitable liquid carrier.

Solutions or suspensions are applied directly to the nasal cavity by conventional means, for example with a dropper, pipette or spray. The compositions may be provided in single or multi-dose form. In compositions intended for administration to the respiratory tract, including intranasal compositions, the compounds, mixtures, or blends will generally have a small particle size, for example of the order of 5 microns or less. Such a particle size may be obtained by means known in the art, for example by micronization.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenges itself, or it can be the appropriate number of any of these in packaged form.

Tablets, capsules and lozenges for oral administration and liquids for oral use are preferred compositions. Solutions or suspensions for application to the nasal cavity or to the respiratory tract are preferred compositions. Transdermal patches comprising creams, lotions, ointments, or salves, and/or other delivery vehicles or delivery systems, for topical administration to the epidermis are preferred. Generally, creams, lotions, ointments, or salves comprising other delivery vehicles or excipients and/or delivery systems for topical delivery are preferred.

Further details on techniques for formulation and administration may be found in the latest edition of Remington's Pharmaceutical Sciences (Mack Publishing Co., Easton, Pa.).

Routes of Administration

The compounds may be administered by any route, including but not limited to oral, sublingual, buccal, ocular, pulmonary, rectal, and parenteral administration, or as an oral or nasal spray (e.g. inhalation of nebulized vapors, droplets, or solid particles). Parenteral administration includes, for example, intravenous, intramuscular, intraarterial, intraperitoneal, intranasal, intravaginal, intravesical (e.g., to the bladder), intradermal, transdermal, topical, or subcutaneous administration. Also contemplated within the scope of the invention is the instillation of a pharmaceutical composition in the body of the patient in a controlled formulation, with systemic or local release of the drug to occur at a later time. For example, the drug may be localized in a depot for controlled release to the circulation, or for release to a local site.

Pharmaceutical compositions of the invention may be those suitable for oral, rectal, bronchial, nasal, pulmonal, topical (including buccal and sub-lingual), transdermal, vaginal or parenteral (including cutaneous, subcutaneous, intramuscular, intraperitoneal, intravenous, intraarterial, intracerebal, intraocular injection or infusion) administration, or those in a form suitable for administration by inhalation or insufflations, including powders and liquid aerosol administration, or by sustained release systems. Suitable examples of sustained release systems include semipermeable matrices of solid hydrophobic polymers containing the compound of the invention, which matrices may be in form of shaped articles, e.g. films or microcapsules.

Without intending to be bound by theory, pterostilbene dosage can be estimated or translated from dosages used in animal studies. Doses from animal studies were translated to human doses by utilizing a Kₘ factor ratio, where Kₘ factors were assigned to each animal model based on their body surface area (Reagan-Shaw, et al., FASEB J. (2007) 22:659-661). The human equivalent dose (HED) is equal to the animal dose multiplied by the ratio: animal's Kₘ/human Kₘ. For example, in the hypercholesterolemic hamster cholesterol study, the hamsters were given 2.5 mg/kg of pterostilbene per day (Rimando et al., 2005). With a Kₘ ratio of 0.135 (with Kₘ values of 5 for hamster/37 for human), the human equivalent dose for this study is 0.337 mg/kg for an adult human, or approx. 25 mg for a 160 pound person per day. Additional studies on diabetes and cognitive function in rats have given human equivalent doses of 118-471 mg/day and 30-118 mg/day respectively (Pari, et al., Life Sci. (2006) 79:641; Joseph, et al., J. Agric. Food Chem. (2008) 56:10544). A pterostilbene 28-day subacute toxicity study in mice showed no local or systemic toxicity at 160 pound human equivalent doses of approximately 125 mg/day, 1.25 g/day, and 12.5 g/day (Ruiz, et al., J. Agric. Food Chem. (2009) 57:3180). Each reference is incorporated herein by reference.

The following is a general study design for human clinical trial to assess efficacy of pterostilbene, in combination with a curcumin, to treat inflammatory bowel disease, or to reduce tumor marker levels and/or tumor size, among other measured endpoints including markers for inflammation and/or oxidative stress. The study type is interventional: a randomized, double-blind, placebo controlled study. Endpoint Classification: Safety/Efficacy Study.

The design of this pilot study will address dosing ranging, safety as well as safety features in observing the addition of pterostilbene and/or a pterostilbene/curcumin to the patient's regimen in obtaining reduction and/or control of symptoms vs. that of placebo.

Inflammatory Bowel Disease (IBD) Study

Mayo Clinic scoring is used which includes stool frequency, rectal bleeding, disease severity based on endoscopy findings and physicians global assessment conducted at 3 month intervals for one year. A short IBD questionnaire is obtained for each visit along with a physical exam of each subject patient, and tests may be run for C-reactive Protein (CRP), complete blood count (CBC), and clinical chemistries. A flexible sigmoidoscopy and biopsy may be obtained at baseline and at 1 year or at time of relapse, along with fecal calprotectin.

IRB approval, full informed consent per patient, and respective clinical trial case report form (CRF) data per visit are obtained.

The purpose of this study is to evaluate whether pterostilbene alone, or a combination of pterostilbene and curcumin compound, will help control the symptoms of inflammation, as well as improve markers for inflammation and/or oxidative stress in patients with IBD meeting inclusion criteria. The investigators will assess the safety of pterostilbene in these patients.

Study Population

Sixty subjects, divided into three groups: 20 per study group. Ages eligible for study: 18 to 80 years, both genders. No healthy volunteers accepted.

Inclusion Criteria:

Irritable Bowel syndrome (IBS) as defined by the Rome III criteria;

patient must be able to swallow study medication;

patient must be able to give consent and follow the treatment plan and be able to answer surveys;

negative serum pregnancy test (females of childbearing potential only) and willing to use an adequate method of contraception throughout the duration of the study;

for those patients above age 40, a colonoscopy within the last 5 years;

no new treatment in the last 2 weeks; and

not receiving any antibiotics in the last 2 weeks.

Exclusion Criteria:

abnormal laboratory values as defined in the protocol;

history of increased gastrointestinal symptoms (“flare”) in the last 3 months;

use of scheduled IBS medications for 4 weeks prior to and during the treatment period (past use of these medications is not an exclusion), or of medications such as azathioprine, methotrexate, or 6-mercaptopurine;

current use (past use of these medications is not an exclusion) of medications or over-the-counter treatments including but not limited to: aspirin, NSAID, botanical treatments (ginger, feverfew, yellow clover, Salix species, Populus species, Betula species, and Gaultheria species), essential fatty acids (flax oil and fish oil); while allowed supplementation includes multivitamin, vitamin D & calcium, folate and vitamin B12, and Iron;

other serious medical conditions such as neurological, liver, kidney, auto- immune or systemic disease, including diabetes, or HIV;

use of anticoagulants or antiplatelet medication, abnormal coagulation or thrombocytopenia, biliary obstruction, symptomatic gallstones, or celiac disease;

ongoing use of medications known to cause or exacerbate symptoms of IBS;

history of gastrointestinal surgery or planned gastrointestinal surgery in the future;

tobacco, alcohol, or illicit drug abuse;

planned surgery during the potential study participation time; and

inability to swallow study medication.

Study Design

The study provides the opportunity to evaluate a method for treatment of inflammatory disorders (i.e., IBD). In other embodiments, the study can provide a method for treating oxidative stress. In other embodiments, the study can provide a method for treating ulcerative colitis, or colorectal cancer. It utilizes a variety of endpoints and outcomes from analysis of molecular markers to standard clinical assessment. This design allows for an economy of effort, testing the putative agent and test combinations first clinically, with associated key endpoint biomarkers for which valuable validation data can be obtained.

Evaluation Criteria for Clinical Measurement:

1. Primary outcome measures: improvement of IBS symptoms and quality of life (QOL) assessed by IBS and QOL questionnaires and a reduction of the initially measured irritable bowel severity score (IBSS) by no less than 25%.

2. Secondary outcome measures: Changes in bowel movement frequency, consistency, frequency of as-needed medication use and bloating as measured by the visual analog scale (VAS), changes in TNF-alpha levels, standard laboratory blood lipid panel.

3. Optional determination of oxidative stress markers (i.e., change in baseline urine-derived markers of stress (e.g., isoprostanes: iPF2-Alpha-III/creat; iPF2-Alpha-VI/creat; 2,3 Dinor-iPF2-Alpha-III/creat)).

Subjects will be divided into three groups or arms: (1) high dose pterostilbene, 125 mg twice daily by mouth, and 250-500 mg curcumin (daily) for 6 to 8 weeks; (2) low dose pterostilbene, 50 mg twice daily by mouth and 250-500 mg curcumin (daily) for 6 to 8 weeks; (3) matching placebo taken twice daily by mouth for 6 to 8 weeks; either one hour before or two hours after a meal. The pterostilbene and the curcumin will be standardized by ChromaDex, Inc. (Irvine, Calif.). All products will be similar in appearance. Blood and urine will be collected at enrollment and final study visits. If the patient's LDL-C or TC is not within the inclusion criteria based on blood drawn at enrollment, the patient will not be allowed to initiate study medication. Patients will be actively participating for 6 to 8 weeks. Patients will be asked to monitor for any symptomatic adverse effects and home blood pressures, as needed. All study visits will consist of brief clinical examination (including vital signs), completed questionnaire (if appropriate), subjective adverse event reporting, and fasting donated blood and urine for clinical laboratory tests. At the enrollment visit, standard recommendations for therapeutic lifestyle interventions will be given to all groups, for example, provided in a printed handout. All blood clinical laboratory tests will be performed at an on-site laboratory. All urine clinical laboratory tests will be performed by an off-site laboratory specializing in oxidative stress analysis. At least 4 mL of urine will be collected during visits, in the morning while the patient is fasting. All policies and standard safeguards for decreasing urine contamination will be followed. Urine samples will be transferred from the collecting laboratory to a −80 ° C. freezer within 3 days of collection. Urine samples will then be shipped frozen on dry ice for analysis. Pill counts will be performed for each study subject to assess compliance.

In an alternative study, 30 patients in remission will receive the pterostilbene/curcumin combination in addition to their present regimen, and 30 patients will be given placebo, and all subjects will undergo baseline and quarterly visits and studies as above noted.

In the above study design, there are no known food effects to be avoided. Without being bound by theory, it is known in the art ingestion of food can interfere with absorption of polyphenolic compounds in the body. It is expected that the study will take into account this food effect during administration of pterostilbene, or the combination of pterostilbene and curcumin.

Useful detection methods for the isoprostane stress markers listed above are available from Kronos Science (Phoenix, Ariz.) utilizing LC/MS/MS methodology to measure isoprostanes in urine and serum. Other useful commercial detection kits for measurement of oxidative stress markers and/or other inflammatory markers or modulators are readily available and well-known. For example, the PAXgene Blood RNA Kit IVD (in vitro diagnostic) is available from Qiagen, Inc. USA (Valencia, Calif.), and may be used for isolation and purification of intracellular RNA from whole blood stabilized in PAXgene Blood RNA Tubes for IVD, followed by applying the known manual or automated technique of reverse transcription-polymerase chain reaction (RT-PCR), or other known PCR techniques, for gene expression and biomarker detection.

Isoprostanes are prostaglandin-like compounds formed from the free radical-catalyzed peroxidation of essential fatty acids (primarily arachidonic acid) without the direct action of cyclooxygenase (COX) enzyme. Isoprostanes are non-classical eicosanoids and possess potent biological activity as inflammatory mediators that augment the perception of pain. Isoprostanes are accurate markers of lipid peroxidation in both animal and human models of oxidative stress, for example, when there is an excessive production of lipid peroxidation products, which may be involved in the development or exacerbation of cancer, isoprostane analysis may measure this process. Isoprostanes may be used in this manner for cardiovascular and neurological diseases as well. Although isoprostanes have a short half-life, some of them have potent biological activities, especially in the lungs and kidney, and may even function in normal physiology. Isoprostanes are useful markers for oxidative stress, and importantly they can be assayed by non-invasive means.

Isoprostanes have been detected in all biological fluids and tissues analyzed to date. There is growing acceptance that measurement of the relatively stable F2-isoprostanes, and 8-IsoPF2α (iPF2-alpha-III) in urine is a reliable non-invasive approach to the determination of the degree of oxidative stress in patients. Normal levels of isoprostanes in healthy humans have been defined, so that the effects of disease states and subsequent therapeutic intervention can be determined. Thus, increased levels of urinary isoprostanes have been measured in many conditions that have been associated with excessive generation of free radicals, including poisoning with carbon tetrachloride, smoking, alcoholism, cirrhosis of the liver, brain degeneration, ischemia-reperfusion injury, atherosclerosis and diabetes. Urinary isoprostane analysis has also been used to assess the efficacy of antioxidants in vivo and to establish the value of antioxidant administration in clinical trials.

The methods described above may be further understood in connection with the following Examples. As used herein, the terms: “HPMC” means hydroxypropyl methylcellulose; “TNF-alpha” means tumor necrosis factor-alpha.

### EXAMPLE 1

In accordance with the clinical trial patient groups, pterostilbene in combination with curcumin is administered orally as follows as either a high daily dose (250 mg of pterostilbene and 500 mg of curcumin) or a low daily dose (100 mg of pterostilbene and 500 mg of curcumin). High dose: 125 mg pterostilbene, 250 mg curcumin, and 25 mg microcrystalline cellulose are combined in a green opaque size 2 HPMC capsule, and administered twice daily. Low dose: 50 mg pterostilbene, 250 mg curcumin, and 100 mg microcrystalline cellulose are combined in a green opaque size 2 HPMC capsule, and administered twice daily. Placebo: 400 mg microcrystalline cellulose in a green opaque size 2 HPMC capsule, administered twice daily.

### EXAMPLE 2

Example 1 is repeated. After about 8 weeks of study monitoring, it is expected that an individual human subject will exhibit lowering of inflammatory and/or oxidative stress markers, as tested in the patient's urine, to a greater extent than a patient administered pterostilbene alone.

### EXAMPLE 2A

Example 1 is repeated. After about 8 weeks of study monitoring, it is expected that an individual human subject will record a reduction in IBSS score of at least 25%, in comparison to a patient administered either pterostilbene alone, or placebo.

### EXAMPLE 3

Equine Study.

Supplementation is provided for each animal in the study group in a daily dosage rate as follows.

Group 1 (n=4): Control or Placebo-Treated.

Group 2 (n=4): Pterostillbene (1.2 grams).

Group 3 (n=4): Pterostillbene (1.2 grams)+curcumin (dosage range: 1 g to 5 g).

Biological Samples will be taken including the following. Potential diagnostic tests are listed.

Serum Tube (10-20 mLs): Chemistry panel (+insulin, mg, sorbitol dehydrogenase=SDH), thiobarbiturate reactive substances (TBARs), Protein Carbonyls, ELISAs for available equine specific analyses.

Heparin Tube (5-10 mLs): determination of reactive oxygen metabolites (d-ROMs) by spectrophotometric assay, biological antioxidant potential (BAP) by spectrophotometric assay, and red blood cells (RBCs) for potential superoxide dismutase (SOD) and Catalase (CAT) Activity to be looked at later, if desired. Plasma is also used for Curcumin and Pterostillbene bioavailability. Free Radical Analytical System (FRAS 4) including two blood tests (the d-ROMs and the BAP tests) is available from Innovatics Laboratories, Inc. (Philadelphia, Pa.).

EDTA Tube (2-6 mLs): complete blood count (CBC). EDTA is ethylenediaminetetraacetic acid used as anticoagulant.

PAXgene Tube (2.5 mLs): inflammatory markers.

Study Design

Horses are fed their normal daily diet of oat and alfalfa hay while being supplemented daily for 6 weeks. During the seventh week, horses are given feed high in peroxide levels while continuing on their respective supplement protocol. This will continue for one more week.

Horses are maintained in individual stalls and walked/trotted for 20-30 min, 3 to 4×/week.

Bioavailability of curcumin and pterostilbene will be determined the first day of supplementation (Day 0), at the end of 6 weeks of supplementation, and again at the end of 7 weeks of supplementation (i.e., after the one week of high peroxide feed). Analytical and pharmacokinetic methods for determination of bioavailability are well known in the art.

Unless otherwise noted, each weekly test collection will include blood draws for the Biological Samples as noted above (except PAXgene draws are not weekly), as well as Clinical Evaluations (including body weight/ body condition score=BCS). The actions described are understood to be performed on each subject horse in the respective study groups.

Day minus-21: Horses moved to pens.

Day minus-7: Preliminary blood drawn for CBC, Chemistry Panel, and FRAS measures, as listed above.

Day 0: Baseline blood collection at fasting. An immediate analysis of the FRAS measures is done to make sure horses are “stable” with regard to these antioxidant/free radical markers. It is expected nothing will have changed since they have been in the pens for several weeks on this particular hay. But if there is a change in the FRAS values, then another week is allowed before redoing the baseline draws. At this point, plasma is obtained for bioavailability analysis, and supplementation is initiated. Further, hay samples are collected and sent for typical analysis.

Day 0: Post-prandial (PP) bioavailability testing. Plasma is obtained at 30 min, 60 min, 120 min (2 hrs), 180 min (3 hrs), 240 min (4 hrs), 360 min (6 hrs), 480 min (8 hrs), 720 min (12 hrs), and 1440 min (24 hrs) following meal+supplement. PAXgene tube is obtained at 2 hrs PP for gene expression.

Day 7: Test collection #1 at fasting.

Day 14: Test collection #2 at fasting.

Day 21: Test collection #3 at fasting. Optional: hay sample submitted for analysis.

Day 28: Test collection #4 at fasting.

Day 35: Test collection #5 at fasting.

Day 42: Test collection #6 at fasting. At this point, plasma is obtained for bioavailability analysis, and hay samples are collected and submitted for analysis.

Day 42: Post-prandial (PP) bioavailability testing. Plasma is obtained at 30 min, 60 min, 120 min (2 hrs), 180 min (3 hrs), 240 min (4 hrs), 360 min (6 hrs), 480 min (8 hrs), 720 min (12 hrs), and 1440 min (24 hrs) following meal+supplement. PAXgene tube is obtained at 2 hrs PP for gene expression.

Day 43 to Day 49: Feed meals with high peroxide levels+Supplement (per study Groups as defined above). During this period, daily blood draws are for d-ROMs and BAP analysis (FRAS measures) only. Optionally, at the same times, or at interim periods, other samples may be obtained to include additional measurements as described above.

Day 49: Test collection #7 at fasting. At this point, plasma is obtained for bioavailability analysis.

Day 49: Post-prandial (PP) bioavailability testing. Plasma is obtained at 30 min, 60 min, 120 min (2 hrs), 180 min (3 hrs), 240 min (4 hrs), 360 min (6 hrs), 480 min (8 hrs), 720 min (12 hrs), and 1440 min (24 hrs) following meal+supplement. PAXgene tube is obtained at 2 hrs PP for gene expression.

After the treatment period, it is expected that horses administered a combination of pterostilbene and curcumin (Group 3) will demonstrate greater improvement in FRAS measures (i.e., measured reduction of oxidative stress), and/or improvement in blood count parameters (indicative of decreased oxidative stress and/or inflammation), and/or reduction in expression of inflammatory markers, compared to horses administered pterostilbene alone (Group 2).

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

I claim:

1. An anti-inflammatory pharmaceutical composition comprising a therapeutically effective amount of pterostilbene, a therapeutically effective amount of curcumin, and a pharmaceutically acceptable carrier.

2. The anti-inflammatory pharmaceutical composition of claim 1, wherein pterostilbene is present in an amount from about 5 mg to about 500 mg, and curcumin is present in an amount from about 100 mg to about 2000 mg.

3. The anti-inflammatory pharmaceutical composition of claim 1, wherein pterostilbene is present in an amount from about 50 mg to about 150 mg.

4. An anti-inflammatory veterinary composition comprising a therapeutically effective amount of pterostilbene, a therapeutically effective amount of curcumin, and an acceptable carrier.

5. The anti-inflammatory veterinary composition of claim 4, wherein pterostilbene is present in an amount from about 1 mg to about 1500 mg, and curcumin is present in an amount from about 10 mg to about 5000 mg.

6. A method of treating an individual for an inflammatory disorder or proliferative disorder, comprising administering to the individual in need of such treatment a pharmaceutical composition including a therapeutically effective amount of pterostilbene, a therapeutically effective amount of a curcumin, and a pharmaceutically acceptable carrier.

7. The method of claim 6, wherein the therapeutically effective amount of pterostilbene for a total daily dose is in a range from about 5 mg to about 500 mg, and the therapeutically effective amount of curcumin for a total daily dose is in a range from about 100 mg to about 2000 mg.

8. The method of claim 6, wherein the therapeutically effective amount of pterostilbene for a total daily dose is in a range from at least about 100 mg to about 250 mg, and the therapeutically effective amount of curcumin for a total daily dose is in a range from at least about 250 mg to about 500 mg.

9. The method of claim 6, wherein the inflammatory disorder is selected from the group consisting of inflammatory bowel disease (IBD), Crohn's disease, gastric esophageal reflux disease (GERD), laminitis, arthritis, sarcoidosis, acne, periodontitis, atherosclerosis, and ulcerative colitis.

10. The method of claim 6, wherein the proliferative disorder is selected from the group consisting of stomach cancer, pancreatic cancer, oral cancer, liver cancer, pre-cancerous polyps, colorectal adenomas (polyps), colon cancer, colorectal cancer, and a cancer of the gastrointestinal tract.

11. The method of claim 6, wherein the individual is a human.

12. The method of claim 8, wherein the individual is a human.

13. The method of claim 6, wherein the individual is a mammal selected from the group consisting of a horse, a bovine species, a pig, a sheep, a goat, a dog, a cat, a rabbit, and a rodent species.

14. A method of reducing oxidative stress or inflammation in a horse, comprising administering to the horse in need of such treatment a composition including a therapeutically effective amount of pterostilbene, a therapeutically effective amount of a curcumin, and an acceptable carrier.

15. The method of claim 14, wherein pterostilbene is present in an amount from about 50 mg to about 1500 mg, and curcumin is present in an amount from about 1000 mg to about 5000 mg.

16. The method of claim 14, wherein the wt./wt. ratio of pterostilbene to curcumin is from about 1:1 to about 1:10 in a daily dose.

17. The method of claim 16, wherein the horse is treated for about 2 weeks to about 7 weeks.

18. The method of claim 16, wherein the horse is treated for about 5 weeks to about 7 weeks.

19. The method of claim 18, wherein blood drawn from the horse indicates a reduction in reactive oxygen metabolites (d-ROMs) by spectrophotometric assay.

20. The method of claim 18, wherein blood drawn from the horse indicates an increase in biological antioxidant potential (BAP) by spectrophotometric assay.
